(19) United States
(12) Patent Application Publication
(10) Pub. No.: US 2024/0385087 A1
(43) Pub. Date: Nov. 21, 2024

(54) METHOD FOR STAINING MITOCHONDRIA

(71) Applicant: TOCRIS COOKSON LIMITED, Bristol (GB)

(72) Inventors: Hannah Jane Maple, Bristol (GB); Paul Wood, Bristol (GB); Darcey Miller, Centennial, CO (US)

(21) Appl. No.: 18/686,693

(22) PCT Filed: Aug. 25, 2022
(86) PCT No.: PCT/GB2022/052189

§ 371 Date: Feb. 26, 2024

(30) Foreign Application Priority Data
Aug. 26, 2021 (GB) 2112199.1

(51) Int. Cl. G01N 1/30; C09B 57/00

(52) U.S. Cl. CPC G01N 1/30; C09B 57/00; G01N 2001/302

(57) ABSTRACT

Methods for staining mitochondria are disclosed involving using a composition containing a cationic species of the formula: (I) wherein at least one of Y and Z is a substituted or unsubstituted azetidine group; X is selected from O, S, SO₂, Se, NR₁₂, P(O)R₁₂, CR₁₃R₁₄, SiR₁₃R₁₄, Te, and GeR₁₃R₁₄. Also disclosed are methods for analysing mitochondria, involving staining a sample of mitochondria. illuminating the stained sample using light of an appropriate wavelength to fluoresce the compound, and observing or imaging a magnified image of the sample.

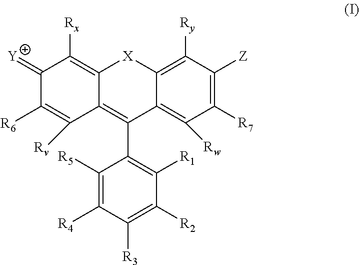

### FIELD OF THE INVENTION

The present invention relates to methods for staining mitochondria, to methods of analysing mitochondria, to methods of detecting mitochondrial conditions and to compounds for use in the detection of mitochondrial conditions.

### BACKGROUND

Functioning mitochondria underpin many critical cellular processes and mitochondrial dysfunction can therefore be a key factor in many diseases. Changes of mitochondrial shape, structure and function sometimes occur in response to changes in energy demand and cellular environment and in some animal (including human) diseases. Mitochondrial diseases may occur because of mutations (inherited or acquired), in mtDNA. Some diseases may also arise from the effects of drugs, infections or other causes.

It is helpful to directly image or observe mitochondria to further understand the nature and pathology of disease or to determine mitochondrial location and morphology for medical or research purposes.

Fluorescent dyes for selectively staining mitochondria are widely used in life sciences research, in applications such as fluorescence microscopy, flow cytometry and high-content screening. Most commercially available mitochondrial stains are organic fluorophores that accumulate in the mitochondrial matrix due to the transmembrane potential, for example MitoTracker™ dyes.

Fluorescent mitochondrial markers (or stains) should combine brightness with high photostability and low toxicity. Photostability is particularly important for studying live-cell mitochondrial morphology because mitochondria are dynamic, undergoing fusion and fission and it is desirable to be able to study this attribute over an extended time period without loss of signal or dye-induced toxicity. Overall brightness (typically measured as the product of the extinction coefficient and quantum yield) influences the concentration of stain that can be used and the final image quality. Increased brightness is a beneficial feature for mitochondrial markers. Dyes for use in imaging mitochondria also need to selectively accumulate in the mitochondria.

There is a need for improved stains that combine increased brightness and photostability with low toxicity.

It is an aim of the present invention to address this need.

### SUMMARY

In a first aspect, there is accordingly provided a method for staining mitochondria, the method comprising: providing a sample containing mitochondria, and incubating the sample in a composition comprising a cationic species of formula (I):

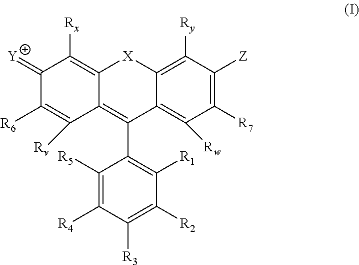

or a solvate, or tautomer thereof; and a counter ion;
wherein:
Y is a substituted or unsubstituted azetidine ring and Z is selected from OR₁₇ or a substituted or unsubstituted azetidine ring;
X is selected from O, S, SO₂, Se, NR₁₂, P(O)R₁₂, CR₁₃R₁₄, SiR₁₃R₁₄, Te, and GeR₁₃R₁₄;
R₁, R₂, R₃, R₄, and Rₛ are each independently selected from H, C₁ to C₈ alkyl, OR₁₅, C(O)OR₁₆, NHC(O)R₁₅, C(O)NHR₁₅ and halo;
Rᵥ, R_{w}, Rₓ, R_{y}, R₆, R₇ are each independently selected from H, C₁ to C₈ alkyl and halo;
R₁₂, R₁₃, R₁₄, and R₁₅ are each independently selected from H, C₁ to C₈ alkyl, optionally substituted aryl or optionally substituted heteroaryl;
R₁₆ is selected from C₁ to C₈ alkyl, optionally substituted aryl or optionally substituted heteroaryl, and
R₁₇ is selected from H, C₁ to C₈ alkyl, optionally substituted aryl or optionally substituted heteroaryl.

In an alternative, a cationic mitochondrial stain of formula 1 may optionally be generated by oxidation within mitochondria or intracellularly of a compound comprising an alternative, reduced form of formula 1, for example as shown in formula (Ib) below:

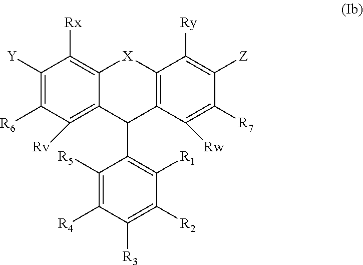

A method according to the first aspect is greatly advantageous because the composition comprising the cationic species provides enhanced photostability with excellent brightness.

The cationic species for use in the invention are greatly advantageous because such delocalized lipophilic cations selectively accumulate in mitochondria due to the negative potential gradient produced by the mitochondrial membrane.

Suitably, at least one of Y and Z is a substituted or unsubstituted azetidine group of formula:

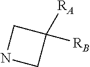

wherein R_{A} and R_{B} are independently selected from H, halo, C₁ to C₈ alkyl, optionally substituted aryl or optionally substituted heteroaryl.

Suitably, when X is SiR₁₃R₁₄, R_{A} and R_{B} may be independently selected from H, Cl, Br, I, C₁ to C₈ alkyl, optionally substituted aryl or optionally substituted heteroaryl. More suitably, when X is SiR₁₃R₁₄, R_{A} and R_{B} may be independently selected from H, C₁ to C₈ alkyl, optionally substituted aryl or optionally substituted heteroaryl.

Optionally, R_{A} and R_{B} may be independently selected from H, Cl, Br, I, C₁ to C₈ alkyl, optionally substituted aryl or optionally substituted heteroaryl.

Thus, suitably the cationic species may be of formula (II):

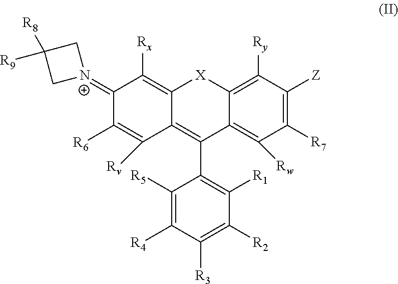

wherein R₈ and R₉ are independently selected from H, halo, C₁ to C₈ alkyl, optionally substituted aryl or optionally substituted heteroaryl.

The counter ion will usually result from the method of synthesis of the cationic species. The counter ion may be changed using ion exchange or other methods as known in the art.

Suitably, the counter ion may be a biologically compatible counter ion. A biologically compatible counter ion is not toxic in use and does not have a substantially harmful effect on biomolecules.

The counter ion may be selected from halide, carboxylate, oxalate, sulfate, alkanesulfonate, arylsulfonate, phosphate, perchlorate, trifluoroacetate, tetrafluoroborate, tetraphenylboride, hexafluorophosphate, nitrate and anions of aromatic or aliphatic carboxylic acids. Suitably, the counter ion may be selected from chloro, acetate or trifluoroacetate.

Incubating the sample may be for a predetermined time, optionally in the range 10 mins to 2hours and at a predetermined temperature, optionally in the range 20° C. to 39° C.

Suitably, the cationic species may be of formula (III):

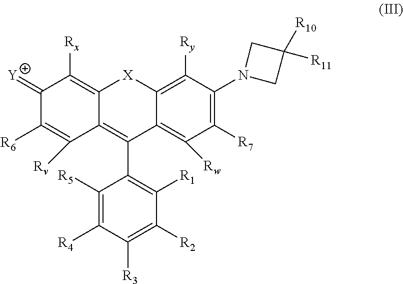

wherein R₁₀ and R₁₁ are independently selected from H, halo, C₁ to C₈ alkyl, optionally substituted aryl or optionally substituted heteroaryl.

Suitably, the cationic species may be of formula (IV):

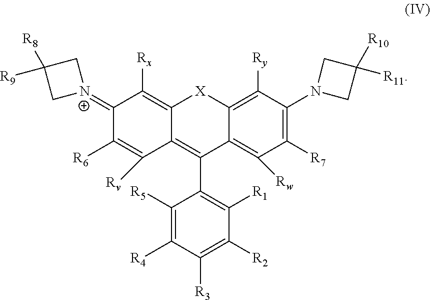

Structurally, the cationic species for use in the method of the invention may comprise an azetidine substituted rosamine (or rosamine analogue wherein X is O, S, SO₂, Se, NR₁₂, P(O)R₁₂, CR₁₃R₁₄, SiR₁₃R₁₄, Te, or GeR₁₃R₁₄) that may have halo, alkyl or other substituents on the pendant phenyl group and elsewhere. In particular, the pendant phenyl group may have an ortho alkyl, optionally an ortho methyl substituent.

Suitably, R₁, R₂, R₃, R₄, R₅, R₆, R₇, R₈, R₉, R₁₀, and R₁₁ may be independently selected from H, fluoro or chloro.

R₁ and/or R₅ may be C₁ to C₈ alkyl. Suitably, R₁ and/or R₅ may be methyl.

Suitably, when X is SiR₁₃R₁₄, R₈, R₉ R₁₀, and R₁₁ may be independently selected from H, Cl, Br, I, C₁ to C₈ alkyl, optionally substituted aryl or optionally substituted heteroaryl. More suitably, when X is SiR₁₃R₁₄, R₈, R₉ R₁₀, and R₁₁ may be independently selected from H, C₁ to C₈ alkyl, optionally substituted aryl or optionally substituted heteroaryl.

Optionally, R₈, R₉ R₁₀, and R₁₁ may be independently selected from H, Cl, Br, I, C₁ to C₈ alkyl, optionally substituted aryl or optionally substituted heteroaryl.

The composition may further comprise at least one organic solvent. The organic solvent may be selected from DMSO, acetone, dimethylformamide, acetonitrile, dioxane, and THF.

Usually, the sample containing mitochondria comprises a tissue sample. The sample containing live mitochondria may be a plant, animal or fungal tissue sample, a sample of plant, animal or fungal cells or isolated plant, animal or fungal mitochondria. Examples of tissue samples include tissue sections, biopsy, blood draws, cytology samples, etc.

The sample containing mitochondria may comprise a sample containing live mitochondria and/or a sample containing mitochondria in live cells.

The sample containing mitochondria may be such that is does not contain substantial numbers of fixed cells, and preferably substantially no fixed cells.

The concentration of the cationic species of formula I in the composition may be in the range 10 nM to 1 μM, preferably 10 nM to 300 nM.

The cationic species may be isotopically labelled. For example, one or more hydrogens may be replaced with deuterium or tritium, or one or more carbons may be replaced with C-13.

Suitably, the cationic species of formula (I) may be selected from species of formulae:

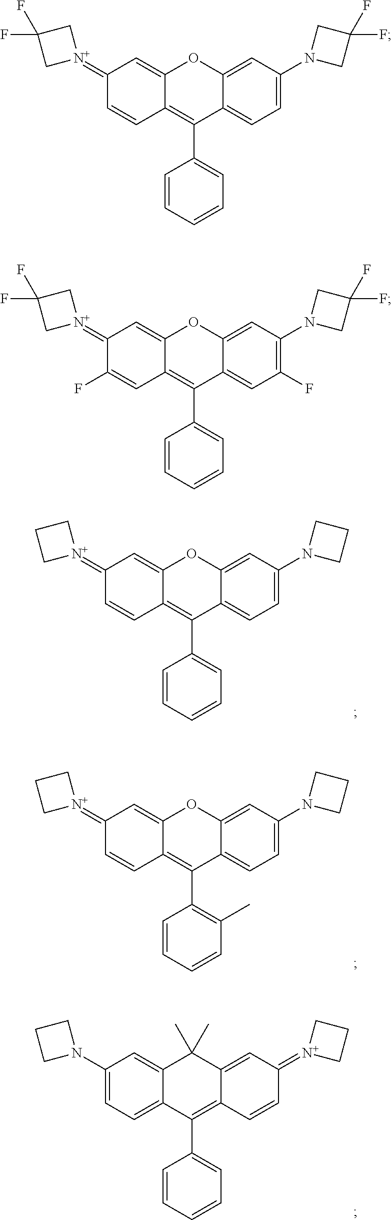 [SMILES: FC1(F)CN(c2ccc3c(-c4ccccc4)c4ccc(=[N+]5CC(F)(F)C5)cc-4oc3c2)C1 ; Fc1cc2c(-c3ccccc3)c3cc(F)c(=[N+]4CC(F)(F)C4)cc-3oc2cc1N1CC(F)(F)C1 ; c1ccc(-c2c3ccc(=[N+]4CCC4)cc-3oc3cc(N4CCC4)ccc23)cc1 ; Cc1ccccc1-c1c2ccc(=[N+]3CCC3)cc-2oc2cc(N3CCC3)ccc12 ; CC1(C)C2=CC(=[N+]3CCC3)C=CC2=C(c2ccccc2)c2ccc(N3CCC3)cc21]

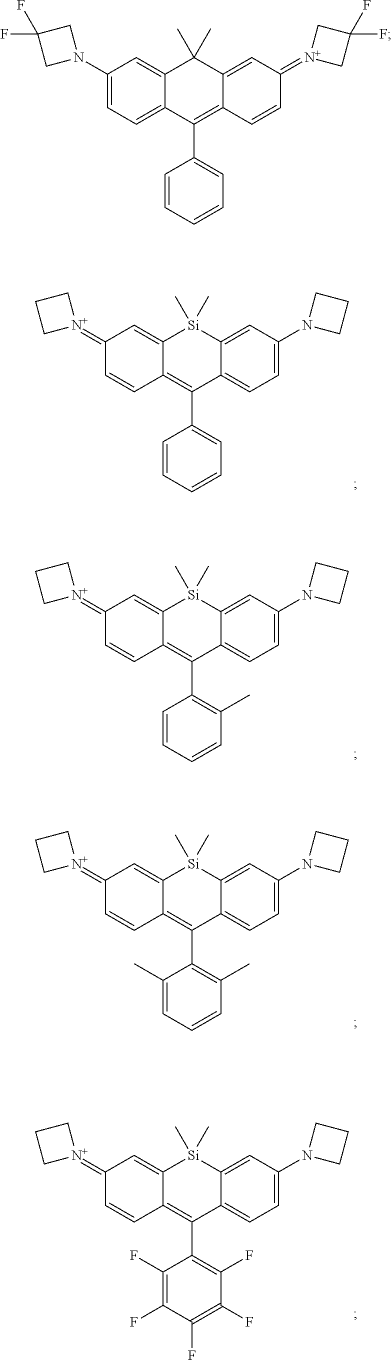 [SMILES: CC1(C)C2=CC(=[N+]3CC(F)(F)C3)C=CC2=C(c2ccccc2)c2ccc(N3CC(F)(F)C3)cc21 ; C[Si]1(C)C2=CC(=[N+]3CCC3)C=CC2=C(c2ccccc2)c2ccc(N3CCC3)cc21 ; Cc1ccccc1C1=C2C=CC(=[N+]3CCC3)C=C2[Si](C)(C)c2cc(N3CCC3)ccc21 ; Cc1cccc(C)c1C1=C2C=CC(=[N+]3CCC3)C=C2[Si](C)(C)c2cc(N3CCC3)ccc21 ; C[Si]1(C)C2=CC(=[N+]3CCC3)C=CC2=C(c2c(F)c(F)c(F)c(F)c2F)c2ccc(N3CCC3)cc21]

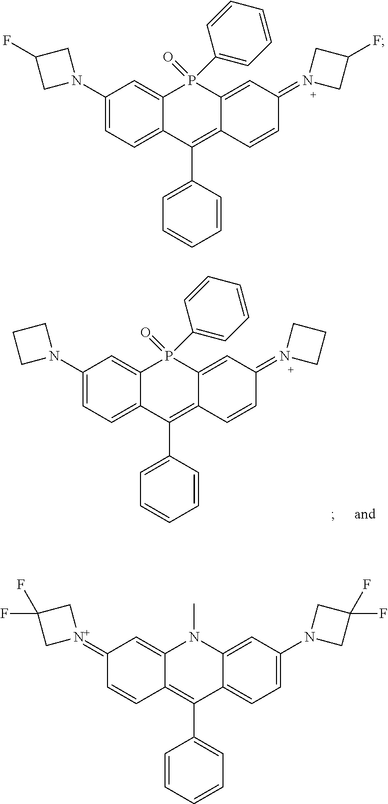

or solvates, or tautomers thereof; and a counter ion.

In a second aspect, there is provided a method of analysing mitochondria, optionally live mitochondria, the method comprising: staining a sample of mitochondria using a method as in the first aspect, illuminating the stained sample using light of an appropriate wavelength to fluoresce the compound, and observing or imaging a magnified image of the sample.

The appropriate wavelength may be in the range 400 nm to 800 nm, preferably 490 nm to 750 nm.

In a third aspect, there is provided a method of detecting or diagnosing a mitochondrial condition comprising staining a sample of mitochondria as in the first aspect and/or analysing a sample of mitochondria as in the second aspect.

The sample of mitochondria may be a plant, animal or fungal tissue sample, a sample of plant, animal or fungal cells or isolated plant, animal or fungal mitochondria.

In a fourth aspect, there is provided a compound comprising a cationic species for use in the detection of a mitochondrial condition, wherein the cationic species is of formula (I):

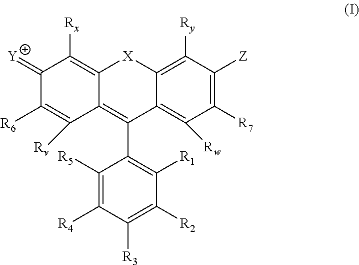

or a solvate, or tautomer thereof; and a counter ion;
wherein:
Y is a substituted or unsubstituted azetidine ring and Z is selected from OR₁₇ or a substituted or unsubstituted azetidine ring;
X is selected from O, S, SO₂, Se, NR₁₂, P(O)R₁₂, CR₁₃R₁₄, SiR₁₃R₁₄, Te, and GeR₁₃R₁₄;
R₁, R₂, R₃, R₄, and R₅ are each independently selected from H, C₁ to C₈ alkyl, OR₁₅, C(O)OR₁₆, NHCOR₁₅, CONHR₁₅ and halo;
Rᵥ, R_{w}, Rₓ, R_{y}, R₆, R₇ are each independently selected from H, C₁ to C₈ alkyl and halo;
R₁₂, R₁₃, R₁₄, and R₁₅ are each independently selected from H, C₁ to C₈ alkyl, optionally substituted aryl or optionally substituted heteroaryl;
R₁₆ is selected from C₁ to C₈ alkyl, optionally substituted aryl or optionally substituted heteroaryl, and
R₁₇ is selected from H, C₁ to C₈ alkyl, optionally substituted aryl or optionally substituted heteroaryl.

The detection or diagnosis may be performed on or in the body of a subject.

Further particular and preferred aspects are set out in the accompanying independent and dependent claims. Features of the dependent claims may be combined with features of the independent claims as appropriate, and in combinations other than those explicitly set out in the claims, as supported by the description.

### DEFINITIONS

“Substituted,” when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

“Optionally substituted” refers to a parent group which may be un-substituted or which may be substituted with one or more substituents. Suitably, unless otherwise specified, when optional substituents are present the optional substituted parent group comprises from one to three optional substituents thus the group may be substituted with 0, 1, 2 or 3 of the optional substituents. Suitably, the group is substituted with 1, 2 or 3 of the optional substituents.

Optional substituents may be selected from C₁₋₈ alkyl, C₁₋₆ alkyl, C₂₋₇ alkenyl, C₂₋₇ alkynyl, C₁₋₁₂ alkoxy, C₅₋₂₀ aryl, C₃₋₁₀ cycloalkyl, C₃₋₁₀ cycloalkenyl, C₃₋₁₀ cycloalkynyl, C₃₋₂₀ heterocyclyl, C₃₋₂₀ heteroaryl, acetal, acyl, acylamido, acyloxy, amidino, amido, amino, aminocarbonyloxy, azido, carboxy, cyano, ether, formyl, guanidino, halo, hemiacetal, hemiketal, hydroxamic acid, hydroxyl, imidic acid, imino, ketal, nitro, nitroso, oxo, oxycarbonyl, oxycarboyloxy, sulfamino, sulfamyl, sulfate, sulfhydryl, sulfinamino, sulfinate, sulfino, sulfinyl, sulfinyloxy, sulfo, sulfonamido, sulfonamino, sulfonate, sulfonyl, sulfonyloxy, uredio groups. In some aspects, the optional substituents are 1, 2 or 3 optional substituents independently selected from OH, C₁₋₈ alkyl, C₁₋₆ alkyl, OC₁₋₁₂ alkyl, and halogen. More suitably, the optional substituents are selected from OH, C₁₋₈ alkyl and OC₁₋₁₂ alkyl; more suitably, the optional substituents are selected from C₁₋₈ alkyl and OC₁₋₁₂ alkyl.

“Independently” or “Independently selected” is used in the context of statement that, for example, “each R₁₆, R₁₇ is independently H, C₁₋₈ alkyl . . . ” and means that each instance of the functional group, e.g., R₁₆, is selected from the listed options independently of any other instance of R₁₆ or R₁₇ in the compound. Hence, for example, H may be selected for the first instance of R₁₆ in the compound; methyl may be selected for the next instance of R₁₆ in the compound; and ethyl may be selected for the first instance of R₁₇ in the compound. C₁₋₈ alkyl: refers to straight chain and branched saturated hydrocarbon groups, having from 1 to 8 carbon atoms, and C₁₋₆ alkyl to straight chain and branched saturated hydrocarbon groups, having from 1 to 6 carbon atoms. Suitably a C₁₋₇ alkyl; suitably a C₁₋₆ alkyl; suitably a C₁₋₅ alkyl; more suitably a C₁₋₄ alkyl; more suitably a C₁₋₃ alkyl. Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, n-octyl and the like.

“Alkylene” refers to a divalent radical derived from an alkane which may be a straight chain or branched, as exemplified by —CH₂CH₂CH₂CH₂—. The alkylene may have the number of carbons as discussed above for alkyl groups.

“Aryl” refers to fully unsaturated monocyclic, bicyclic and polycyclic aromatic hydrocarbons having at least one aromatic ring. Aryl groups as used herein are preferably “C₅₋₂₀ Aryl” a fully unsaturated monocyclic, bicyclic and polycyclic aromatic hydrocarbons having at least one aromatic ring and having a specified number of carbon atoms that comprise their ring members (e.g., C₅₋₂₀ aryl refers to an aryl group having from 5 to 20 carbon atoms as ring members). The aryl group may be attached to a parent group or to a substrate at any ring atom and may include one or more non-hydrogen substituents unless such attachment or substitution would violate valence requirements. Suitably, a is selected from a C₆₋₁₂ aryl, more suitably, a C₆₋₁₀ aryl. Examples of aryl groups include phenyl.

“Halogen” or “halo”: refers to a group selected from F, Cl, Br, and I. The halogen or halo may be F or Cl. In some aspects, the halogen may be F. In other aspects, suitably the halogen is Cl, Br or I; preferably Cl.

“Heteroaryl” refers to unsaturated monocyclic or bicyclic aromatic groups. Preferably heteroaryl is “C₅₋₁₀ heteroaryl” or “5- to 10-membered heteroaryl” an unsaturated monocyclic or bicyclic aromatic group comprising from 5 to 10 ring atoms, whether carbon or heteroatoms, of which from 1 to 5 are ring heteroatoms. Suitably, any monocyclic heteroaryl ring has from 5 to 6 ring atoms and from 1 to 3 ring heteroatoms. Suitably each ring heteroatom is independently selected from nitrogen, phosphorus, oxygen, sulfur and silicon. The bicyclic rings include fused ring systems and, in particular, include bicyclic groups in which a monocyclic heterocycle comprising 5 ring atoms is fused to a benzene ring. The heteroaryl group may be attached to a parent group or to a substrate at any ring atom and may include one or more non-hydrogen substituents unless such attachment or substitution would violate valence requirements or result in a chemically unstable compound.

Examples of monocyclic heteroaryl groups include, but are not limited to, those derived from:
N₁: pyrrole, pyridine;
O₁: furan;
S₁: thiophene;
N₁O₁: oxazole, isoxazole, isoxazine;
N₂O₁: oxadiazole (e.g., 1-oxa-2,3-diazolyl, 1-oxa-2,4-diazolyl, 1-oxa-2,5-diazolyl, 1-oxa-3,4-diazolyl);
N₃O₁: oxatriazole;
N₁S₁: thiazole, isothiazole;
N₂: imidazole, pyrazole, pyridazine, pyrimidine, pyrazine;
N₃: triazole, triazine; and,
N₄: tetrazole.

Examples of heteroaryl groups which comprise fused rings, include, but are not limited to, those derived from:
O₁: benzofuran, isobenzofuran;
N₁: indole, isoindole, indolizine, isoindoline;
S₁: benzothiofuran;
N₁O₁: benzoxazole, benzisoxazole;
N₁S₁: benzothiazole;
N₂: benzimidazole, indazole;
O₂: benzodioxole;
N₂O₁: benzofurazan;
N₂S₁: benzothiadiazole;
N₃: benzotriazole; and
N₄: purine (e.g., adenine, guanine), pteridine;

As used herein, “solvate” refers to a complex of variable stoichiometry formed by a solute and a solvent. Solvates may be formed for crystalline compounds wherein solvent molecules are incorporated into the crystalline lattice during crystallization. The incorporated solvent molecules can be water molecules or non-aqueous molecules, such as but not limited to, ethanol, isopropanol, dimethyl sulfoxide, acetic acid, ethanolamine, and ethyl acetate molecules.

“Tautomer,” refers to a structural isomer of a compound that readily interconverts to another isomer.

“Fixed cells” refers to cells that have undergone a fixing process to substantially end biochemical reactions within the cells. References to “fixed mitochondria” refer to mitochondria that are or were present in cells that have undergone the fixing process or mitochondria that have undergone a fixing process in order to substantially end biochemical reactions within the mitochondria.

In this specification, “live mitochondria” refers to mitochondria that are functioning in the sense that there is a mitochondrial membrane potential and/or the membrane has not been substantially ruptured.

“Mitochondrial conditions,” as used herein are mitochondrial diseases or conditions involving or that may lead to mitochondrial dysfunction where mitochondria fail to produce enough energy for the body or parts of the body to function properly. Mitochondrial conditions may be chronic, and genetic. Mitochondrial dysfunction occurs when the mitochondria are affected by another disease or condition. Conditions that may lead to such mitochondrial dysfunction include Alzheimer's disease, muscular dystrophy, Lou Gehrig's disease, diabetes and cancer. Mitochondrial conditions/diseases include: Kearns-Sayre syndrome, Leber's hereditary optic neuropathy, Progressive external ophthalmoplegia, Mitochondrial encephalomyopathy with lactic acidosis and stroke-like episodes (MELAS), and Myoclonic epilepsy with ragged red fibres (MERRF).

The term “subject” as used herein refers to a human or non-human animal, suitably a mammal. Examples of non-human mammals include livestock animals such as sheep, horses, cows, pigs, goats, rabbits and deer; and companion animals such as cats, dogs, rodents, and horses.

As used herein the term “comprising” means “including at least in part” and is inclusive or open ended. When interpreting each statement in this specification that includes the term “comprising,” features, elements and/or steps other than that or those prefaced by the term may also be present. Related terms such as “comprise” and “comprises” are to be interpreted in the same manner. It should be understood that while various aspects in the specification are presented as “comprising,” this includes aspects that “consist essentially of” or “consist of” that aspect.

The term “consisting essentially of” limits the scope of a claim to the specified materials or steps “and those that do not materially affect the basic and novel characteristic(s)” of the claimed invention. When the phrase “consisting essentially of” appears in a clause of the body of a claim, rather than immediately following the preamble, it limits only the element set forth in that clause.

The term “consisting of” excludes any element, step, or ingredient not specified in the claim; “consisting of” defined as “closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

### BRIEF DESCRIPTION OF THE DRAWINGS

Embodiments of the present invention will now be described further, with reference to the accompanying drawings, in which:

FIG. 1 shows chemical structures of compounds used in the invention.

FIG. 2 shows images of HeLa cells pre-treated with COMPOUND 1 or a comparator, and subsequently treated with compounds (oligomycin and CCCP) that hyper-and de-polarize the mitochondrial membrane, respectively.

FIG. 3 shows time-course images of HeLa cells incubated with either COMPOUND 1 or a comparator and a graph of normalized intensity with time for both COMPOUND 1 and a comparator.

FIG. 4 shows time-course images of HeLa cells incubated with COMPOUND 1,COMPOUND 2 or COMPOUND 3 and the corresponding graph of normalized intensity with time.

FIG. 5 shows time-course images of HeLa cells incubated with COMPOUND 3 or COMPOUND 4 and the corresponding graph of normalized intensity with time.

FIG. 6 shows images of Hela cells incubated with different concentrations of COMPOUND 1 or a comparator. FIG. 7 shows normalised intensity (a.u) against wavelength for emission and absorption of COMPOUND 1.

FIG. 8 shows normalised intensity (a.u) against wavelength for emission and absorption of COMPOUND 4.

### DESCRIPTION OF THE EMBODIMENTS

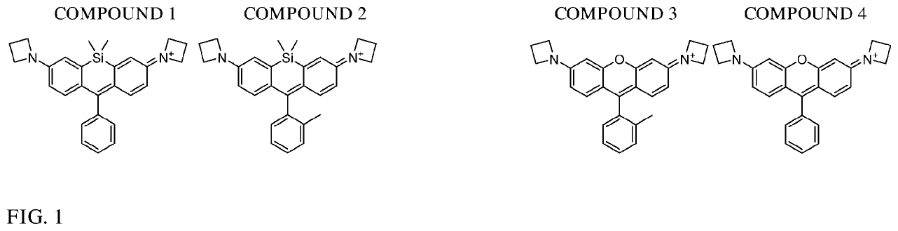

FIG. 1 shows chemical structures of cationic species of COMPOUND 1, COMPOUND 2,COMPOUND 3 and COMPOUND 4 for use in the invention. Azetidine substituted rosamine (or Si rosamine) with/without an ortho-methyl substituent. The compounds shown in FIG. 1 have been synthesized and have undergone tests to demonstrate their utility in the context of the described invention.

The compounds outlined in FIG. 1 cover two core ‘series’ that are primarily defined by distinct excitation/emission profiles. Further compounds with cationic species as in formula I may have different excitation/emission wavelengths.

COMPOUND 1 was extensively tested and shown to be a mitochondrial stain that localizes specifically to the mitochondria due to the charge potential across the mitochondrial membrane (the same mechanism as an existing commercially available comparator compound ‘MitoTracker DeepRed’™).

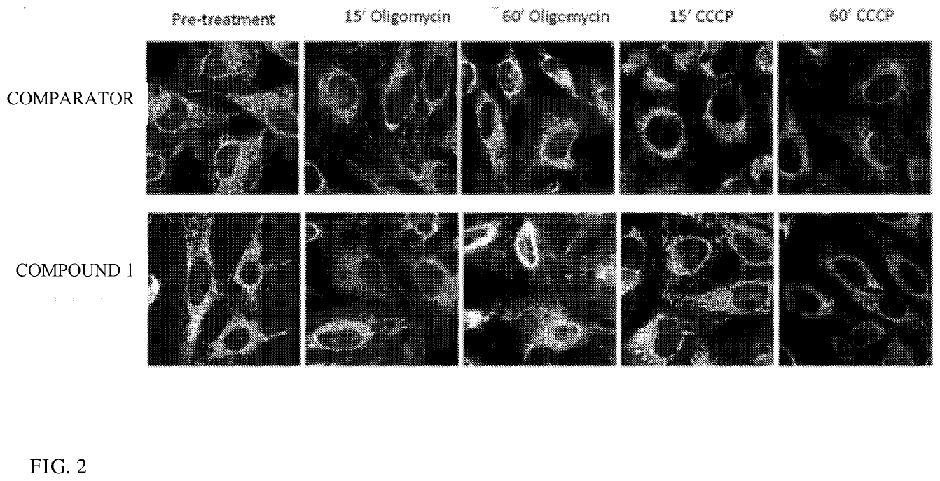

FIG. 2 shows HeLa cells incubated with the comparator compound (50 nM) or COMPOUND 1 (50 nM) for 60 mins, followed by treatment with Oligomycin (5 μg/mL) or CCCP (10 μM) for up to 60 minutes: the images are taken at specified time points. FIG. 2 demonstrates that COMPOUND 1 localizes to the mitochondria via the same mechanism as the comparator, since hyper-and de-polarizing the mitochondrial membrane with oligomycin and CCCP treatment (respectively) causes accumulation and dispersion (respectively) of both the comparator and COMPOUND 1.

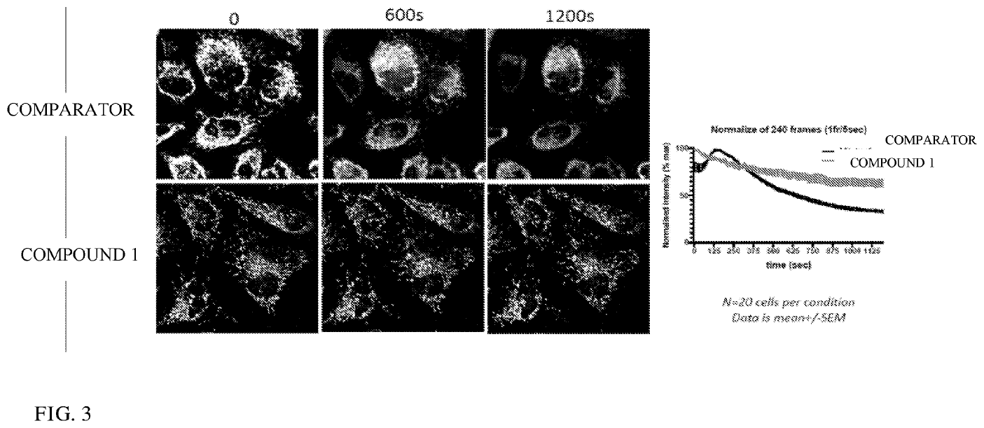

FIG. 3 shows HeLa cells incubated with 50 nM of the comparator or COMPOUND 1 for 60 mins, followed by live imaging with images taken every 5 seconds for 240 frames, shown at specified time points. There is a clear increase in non-specific free dye in the comparator images that accumulates in the nucleus and cytoplasm over time (thus the signal: noise ratio for the mitochondria was reduced)-this results in an apparent increase in intensity over the first 5 minutes, but a significant reduction in overall intensity is seen for the comparator over the full imaging period (20 minutes @1 fr/5 sec). The graph shows time versus normalized intensity-NB: 100% is taken as the maximum intensity peak during movie-hence the comparator probe values start lower, then peak as apparent intensity increases due to non-specific accumulation of dye. Within this time frame, COMPOUND 1 intensity drops by ˜25% during the imaging period compared to the comparator which drops by 65%. COMPOUND 1 remains clearly localized at the mitochondria, whereas the comparator is no longer located in the mitochondria and the cells have begun to contract, indicating (photo)-toxicity. FIG. 3 demonstrates the improved performance of this invention versus the comparator. COMPOUND 1 shows significantly improved photostability and localization within the mitochondria over time, with no apparent toxicity, while the comparator is less photostable, does not remain in the mitochondria over time and exhibits some (photo)-toxicity after prolonged imaging.

In some compounds, an ortho-methyl group may improve the quantum yield (i.e., brightness. The ortho-methyl substituent was present in COMPOUND 2 and COMPOUND 3and absent in the corresponding matched-pair compounds COMPOUND 1 and COMPOUND 4 (FIG. 1).

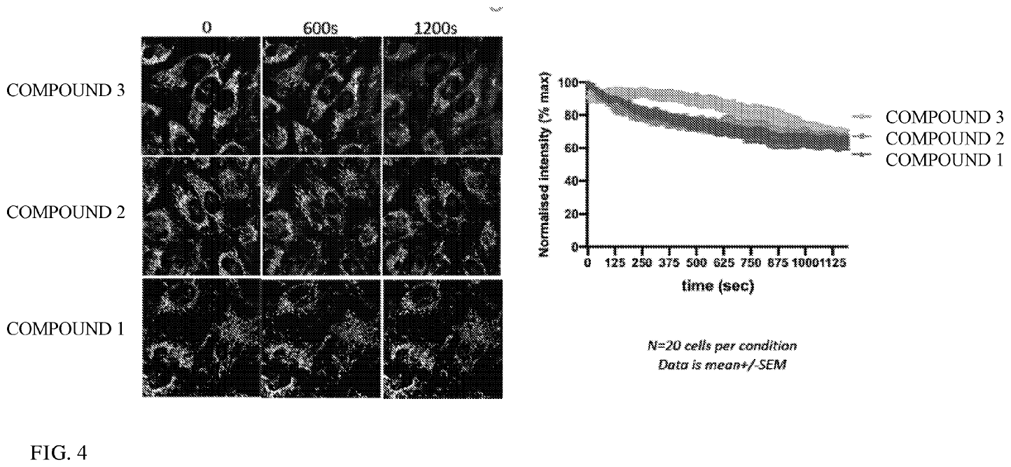

FIG. 4 shows HeLa cells incubated with 50 nM COMPOUND 1, COMPOUND 2 or COMPOUND 3 for 60 mins, followed by live imaging with images taken every 5 seconds for 240 frames, shown at specified time points. COMPOUND 1 and COMPOUND 2 perform very similarly in terms of signal over time; however COMPOUND 1 appears to mark the mitochondria more clearly than COMPOUND 2 over time. COMPOUND 3 showed retention of signal during the first 10 mins of imaging, but then the signal rapidly drops and higher non-specific cytoplasmic background (and lower mitochondrial labelling) was seen.

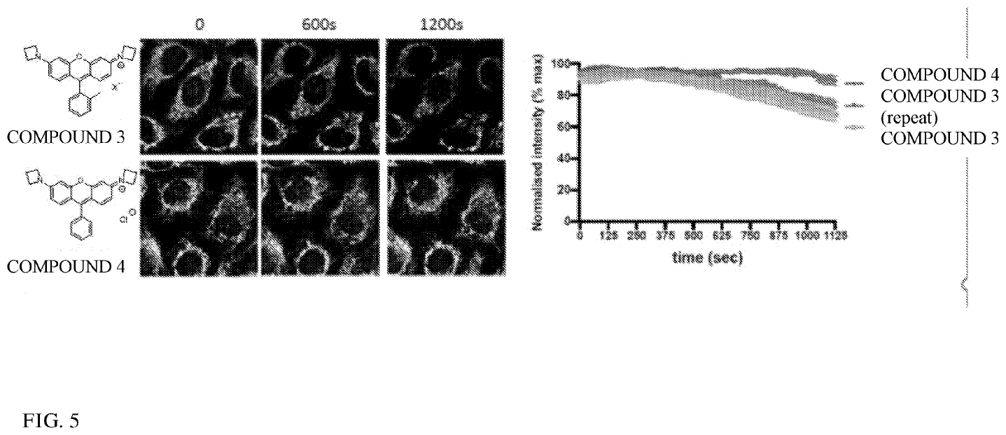

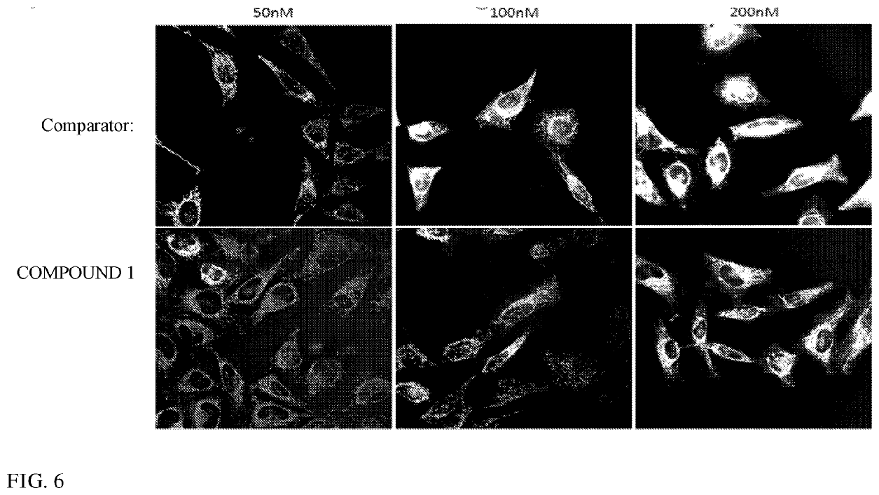

FIG. 5 shows HeLa cells incubated with 50 nM COMPOUND 3 (two samples, repeats) for 60 mins, followed by live imaging with images taken every 5 seconds for 240 frames, shown at specified time points. COMPOUND 3 show retention of signal during the first 10 mins of imaging, but then the signal rapidly drops and higher non-specific cytoplasmic background (and lower mitochondrial labelling) was seen. Data from two samples of COMPOUND 3,measured at different timepoints is overlaid in the graph (right). COMPOUND 4 shows higher signal retention over time than COMPOUND 3 and appears to remain faithfully localized to mitochondria over the entire imaging period with no observable phototoxicity effects.

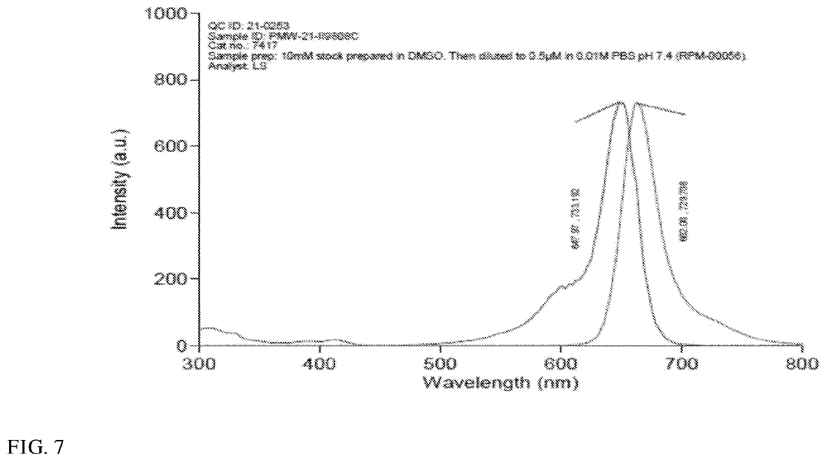

FIG. 6 shows optimization of concentrations required for imaging. HeLa cells were incubated with the comparator or COMPOUND 1 for 60 mins at indicated doses and imaged at the same laser power/settings. NOTE: no washout step to remove unbound comparator was performed (manufacturer recommends this) to enable direct comparison with COMPOUND 1. Some toxicity is observed with 100 nM of comparator. Both COMPOUND 1 and the comparator give excellent results at 50 nM.

FIG. 7 shows normalised intensity (a.u) against wavelength for emission and absorption of COMPOUND 1:1-(7-(azetidin-1-yl)-5,5-dimethyl-10-phenyldibenzo[b,e]silin-3 (5H)-ylidene)azetidin-1-ium chloride

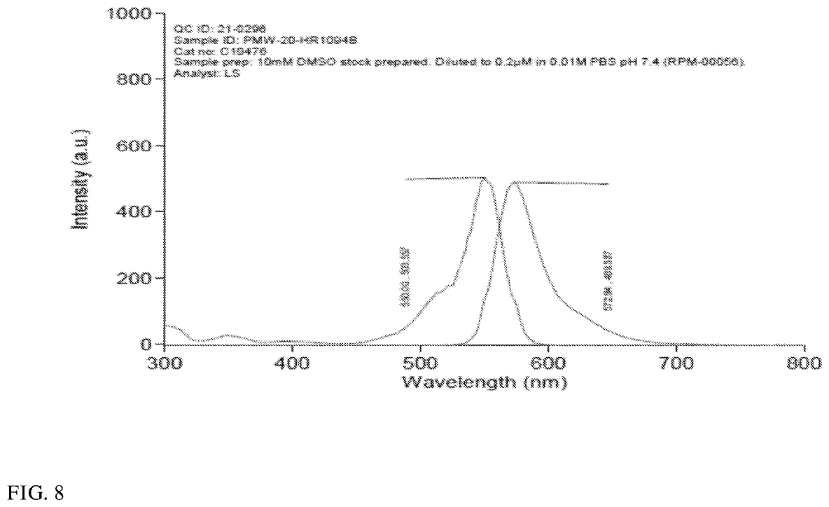

FIG. 8 shows normalised intensity (a.u) against wavelength for emission and absorption of COMPOUND 4:1-(6-(azetidin-1-yl)-9-phenyl-3H-xanthen-3-ylidene)azetidin-1-ium chloride

### Confocal Microscopy

Mitochondrial stains were diluted to working concentrations from 10 mM DMSO stock solutions into DMEM containing 10% FCS and 25 mM HEPES. Solutions were incubated with HeLa cells at 37° C. in a humidified 5% CO₂ incubator for the indicated period of time prior to imaging, typically without a washout step (though a washout step can be performed). A Nikon A1R TiE confocal laser scanning microscope equipped with environmental chamber (37° C.) was used for live-cell imaging, employing 561 nm and 640 nm diode laser lines, a Nikon A1R Plan APO VC 60x Oil lens (NA 1.4), pinhole at 1AU. The images were acquired using NIS Elements software and processed using ImageJ.

### General Chemistry Methods

All reagents and solvents were purchased from commercial sources and used without further purification. Nuclear magnetic resonance spectra were recorded on a Bruker Avance III HD spectrometer operating at 400 MHz for ¹H NMR and 100 MHz for ¹³C NMR. ¹H NMR and ¹³C NMR chemical shifts (δ) are reported in parts per million (ppm) and are referenced to residual protium in solvent and to the carbon resonances of the residual solvent peak respectively.

Purification by flash chromatography was performed using pre-packed silica gel columns and either a Buchi Reveleris, a Biotage Isolera or a Biotage Selekt system. Analytical thin layer chromatography was performed on glass plates pre-coated with silica gel (Analtech, UNIPLATE™ 250 μm/UV254), with visualization being achieved using UV light (254 nm) and/or by staining with alkaline potassium permanganate dip.

Reaction monitoring LC-MS analyses were conducted using Agilent InfinityLab LC/MSD systems. High resolution mass spectral (HRMS) data was collected using an Agilent 6545 LC/Q-TOF system.

Normalized absorption and fluorescence emission spectra were recorded in 10 mM PBS pH 7.3 at the concentration noted for each sample following dilution of a DMSO stock solution. Absorption spectra were recorded with an Agilent Cary 60 UV-Vis spectrophotometer using genuine precision quartz cells from Lovibond with a 1 cm path length. Fluorescence spectra were recorded on an Agilent Cary Eclipse Fluorescence Spectrophotometer using high precision Quartz Suprasil cells from Hellma Analytics and a 1 cm path length.

### EXAMPLES

The invention is further illustrated by the following Examples.

### Example 1-1-(7-(azetidin-1-yl)-5,5-dimethyl-10-phenyldibenzo [b,e]silin-3 (5H)-ylidene)azetidin-1-ium acetate

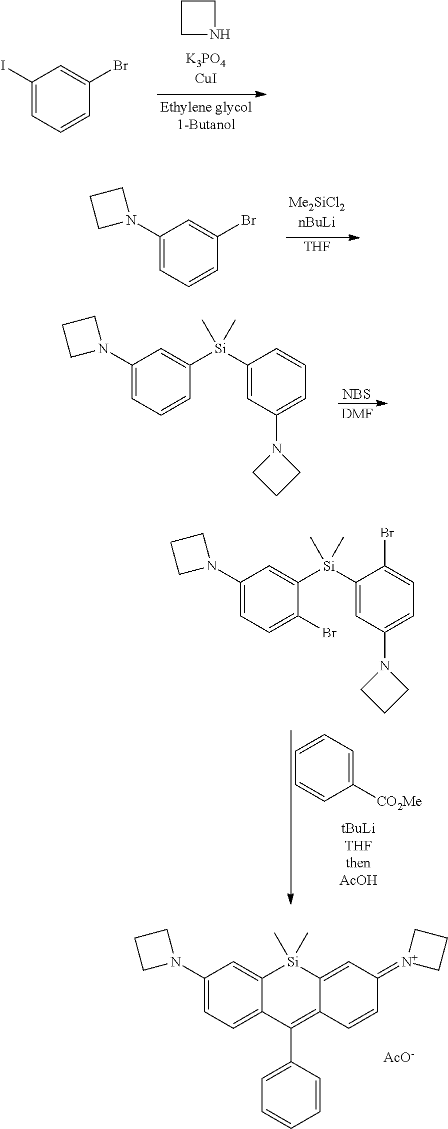

### Synthesis of 1-(3-bromophenyl)azetidine

3-Bromoiodobenzene (30 g, 106 mmol), azetidine (7.27 g, 127 mmol) and K₃PO₄ (67.5 g, 318 mmol) were combined with ethylene glycol (14.2 mL) and 1-butanol (150 mL) in a round bottom flask. The flask was sealed and evacuated/backfilled three times with nitrogen. CuI (2.02 g, 10.6 mmol) was subsequently added and the flask was again sealed and evacuated/backfilled with nitrogen three times. The mixture was then heated at 100° C. under an atmosphere of N₂ for 4 h. After cooling to RT, saturated aqueous NH₄Cl and EtOAc were added with stirring until there were no solids remaining. The layers were separated and the aqueous was extracted twice with EtOAc. The combined organic layers were washed with brine, then dried (MgSO₄) and filtered and the solvent was removed in vacuo. The residue was further dried under high vacuum. The crude product was purified by flash chromatography (0 to 10% Et₂O/PE) to give the title compound as a pale-yellow oil (18.2g, 81%).

¹H NMR (CDCl₃, 400 MHZ) δ 7.04 (1H, t), 6.85-6.80 (1H, m), 6.55 (1H, t), 6.36-6.31 (1H, m), 3.87 (4H, t), 2.37 (2H, p).

### Synthesis of bis(3-(azetidin-1-yl) phenyl)dimethylsilane

A solution of 1-(3-bromophenyl) azetidine (12.6 g, 59.5 mmol) in THF (115 mL) was cooled to −78° C. under nitrogen. A solution of n-butyllithium in hexane (2.5 M, 23.8 mL, 59.5 mmol) was slowly added so that the internal temperature was maintained below −60° C. during the addition. The reaction mixture was subsequently stirred at −78° C. for 30 min. A solution of dichlorodimethylsilane (3.20 g, 24.8 mmol) in THF (10 mL) was then added at a rate such that the internal temperature was kept below −60° C. The cooling bath was removed, and the reaction was stirred at room temperature for 3 h. It was subsequently quenched with saturated aqueous NH₄Cl (20 mL), diluted with water, and extracted twice with EtOAc. The combined organic extracts were washed with brine, dried over anhydrous MgSO₄, filtered, and concentrated in vacuo. The resulting residue was co-evaporated twice with Et₂O and purified by flash chromatography (0 to 30% Et₂O/PE) to give the title compound as a colourless oil (8.00 g, 84%).

¹H NMR (CDCl₃, 400 MHZ) δ 7.20 (2H, t), 6.90 (2H, d), 6.61 (2H, d), 6.46 (2H, ddd), 3.86 (8H, t), 2.34 (4H, p), 0.51 (6H, s).

### Synthesis of bis(5-(azetidin-1-yl)-2-bromophenyl)dimethylsilane

N-Bromosuccinimide (7.45 g, 41.9 mmol) was added in portions over 5 minutes to a solution of bis(3-(azetidin-1-yl)phenyl)dimethylsilane (6.75 g, 20.9 mmol) in DMF (120 mL). The resulting mixture was stirred for 5 days. Following removal of the solvent in vacuo, the resulting residue was diluted with water and extracted with EtOAc and then with DCM. The combined organic layers were washed with water and brine, then dried (MgSO₄) and filtered and the solvent was removed in vacuo. The crude product was purified by recrystallisation from EtOAc to give the title compound as a white solid (5.69 g, 57%).

¹H NMR (CDCl₃, 400 MHZ) δ 7.31 (2H, d), 6.51 (2H, d), 6.31 (2H, dd), 3.81 (8H, t), 2.36 (4H, p), 0.71 (6H, s).

### Synthesis of 1-(7-(azetidin-1-yl)-5,5-dimethyl-10-phenyldibenzo[b,e]silin-3 (5H)-ylidene)azetidin-1-ium acetate

A solution of t-BuLi in pentane (1.7 M, 1.96 mL) was added dropwise to a cooled (−78° C.) solution of bis(5-(azetidin-1-yl)-2-bromophenyl)dimethylsilane (0.40 g, 0.83 mmol) in THF (40 mL). After stirring for 20 minutes, the reaction mixture was warmed to −20° C. and a solution of methyl benzoate (0.25 g, 1.83 mmol) in THF (7 mL) was added over 20 minutes, maintaining the internal temperature below −20° C. over the course of the addition. The resulting mixture was allowed to warm to room temperature and was stirred overnight. The following day, saturated aqueous NH₄Cl and water were added and the product was extracted twice with EtOAc The combined organic layers were washed with brine, then dried (MgSO₄) and filtered and the solvent was removed in vacuo. The resulting residue was dissolved in MeOH (20 mL) and AcOH (0.2 mL) was added. After stirring for 15 minutes, the mixture was concentrated in vacuo. The crude product was purified by flash chromatography (1% MeOH in DCM (+1% AcOH) to 15% MeOH in DCM (+1% AcOH) to give the title compound as a blue solid (0.27 g, 69%).

¹H NMR (MeOD, 400 MHZ) δ 7.60-7.52 (3H, m), 7.28-7.21 (2H, m), 7.08 (2H, d), 6.94 (2H, d), 6.34 (2H, dd), 4.36 (8H, t), 2.55 (4H, p), 1.96 (3H, s), 0.55 (6H, s).

LC/MS (ES+): m/z 409.3 (100%, M⁺).

### Example 2-1-(7-(azetidin-1-yl)-5,5-dimethyl-10-phenyldibenzo[b,e]silin-3 (5H)-ylidene)azetidin-1-ium chloride

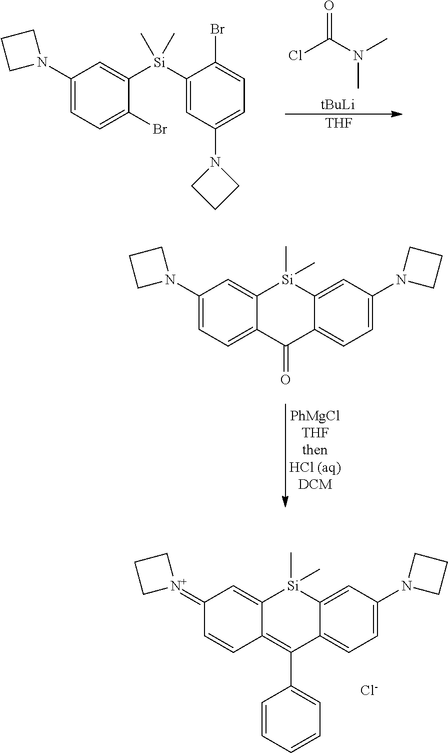

### Synthesis of 3,7-di(azetidin-1-yl)-5,5-dimethyldibenzo[b,e]silin-10 (5H)-one

A solution of t-BuLi in pentane (1.7 M, 10.9 mL) was added dropwise to a cooled (−78° C.) solution of bis(5-(azetidin-1-yl)-2-bromophenyl)dimethylsilane (2.00 g, 4.16 mmol) in THF (160 mL). After stirring for 20 min, N,N-dimethylcarbamoyl chloride (0.49 g, 4.58 mmol) was added dropwise over 20 minutes. The reaction mixture was allowed to room temperature and was stirred overnight. The following day it was diluted with saturated aqueous NH₄Cl and THF and the product was extracted twice with additional THF. The combined organic layers were washed with brine, then dried (MgSO₄) and filtered and the solvent was removed in vacuo. The resulting residue was dissolved in DCM, dried (MgSO₄) and filtered and the solvent was removed in vacuo. The crude product was purified by flash chromatography (0 to 15% MeOH in DCM) followed by trituration with hot EtOAc to give the title compound as a yellow solid (1.12 g, 77%).

¹H NMR (CDCl₃, 400 MHZ) δ 8.36 (2H, d), 6.53 (2H, dd), 6.48 (2H, d), 4.03 (8H, t), 2.44 (4H, p), 0.43 (6H, s).

HRMS (ESI) calcd. for C21H24N2OSi [M+H]⁺, 349.1660, found 349.1733.

### Synthesis of 1-(7-(azetidin-1-yl)-5,5-dimethyl-10-phenyldibenzo[b,e]silin-3 (5H)-ylidene)azetidin-1-ium chloride

A solution of phenylmagnesium chloride in THF (2M, 1.40 mL) was added to a stirred suspension of 3,7-di (azetidin-1-yl)-5,5-dimethyldibenzo[b,e]silin-10 (5H)-one (0.75 g, 2.15 mmol) in THF (25 mL). After stirring for 1 h, the reaction mixture was diluted with DCM and saturated aqueous NH₄Cl. The layers were separated and the aqueous was further extracted three times with DCM. The combined organic layers were dried (MgSO₄), filtered and the solvent was removed in vacuo. The resulting residue was dissolved in DCM and 1 drop of 2 M aqueous HCl was added. The mixture was subsequently concentrated in vacuo and then co-evaporated with firstly MeOH and then with DCM. The crude product was purified by flash chromatography (5 to 20% MeOH in DCM) followed by drying in the vacuum oven to give the title compound as a blue/green solid (0.15 g, 10%).

¹H NMR (CDCl₃, 400 MHZ) δ 7.52-7.49 (3H, m), 7.21-7.18 (2H, m), 7.05 (2H, d), 6.88 (2H, d), 6.25 (2H, dd), 4.49-4.38 (8H, m), 2.62 (4H, p), 0.60 (6H, s).

LC/MS (ES⁺): m/z 409.3 (100%, M⁺).

### Example 3-1-(6-(azetidin-1-yl)-9-phenyl-3H-xanthen-3-ylidene)azetidin-1-ium chloride

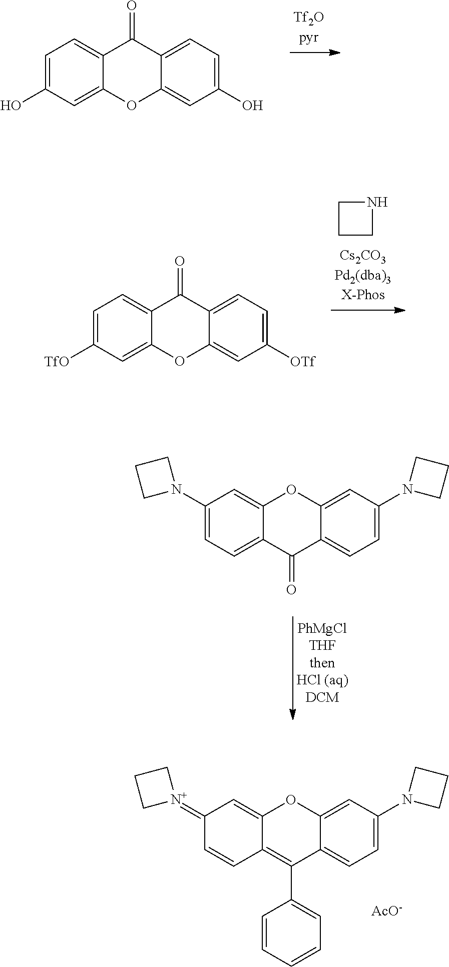

### Synthesis of 9-oxo-9H-xanthene-3,6-diyl bis(trifluoromethanesulfonate)

Pyridine was slowly added to a cooled (0° C.) suspension of 3,6-dihydroxyxanthen-9-one (3.65 g, 16.0 mmol) in DCM (60 mL). The reaction mixture was stirred for 5 minutes before trifluoromethanesulfonic anhydride (13.5 g, 47.8 mmol) was added dropwise keeping the internal temperature of the mixture below 15° C. The reaction mixture was allowed to warm to room temperature and was stirred overnight. The following day, water and DCM were added and the layers were separated. The organic layer was washed with water, 1 M aqueous HCl and brine then dried (MgSO₄) and filtered and the solvent was removed in vacuo. The crude product was purified by precipitation of the product from a DCM solution by treatment with petroleum ether to give the title compound as a white solid (5.63 g, 72%).

¹H NMR (CDCl₃, 400 MHZ) δ 8.46 (2H, d), 7.50 (2H, d), 7.36 (2H, dd).

### Synthesis of 3,6-di(azetidin-1-yl)-9H-xanthen-9-one

A mixture of 9-oxo-9H-xanthene-3,6-diyl bis (trifluoromethanesulfonate (5.63 g, 11.4 mmol), Pd₂ (dba)₃ (1.05 g, 1.15 mmol), XPhos (1.49 g, 3.4 mmol) and Cs2CO3 (17.9 g, 54.9 mmol) in a round bottom flask was evacuated/backfilled with nitrogen (X3). To this mixture was added azetidine (1.44 g, 25.2 mmol) and 1,4-dioxane (65 ml) and the flask was again evacuated/backfilled three times with nitrogen. The flask was then inserted into a pre-heated metal heating block and stirred at 105° C. for 8 h. After cooling to room temperature, the reaction mixture was diluted with DCM/water and the layers were separated. The aqueous layer was extracted twice with additional DCM. The combined organic layers were dried (MgSO₄), filtered and the solvent was removed in vacuo. The crude product was purified by flash chromatography (10 to 20% MeOH in DCM) followed by trituration with acetone to give the title compound as a yellow solid (0.46 g, 13%).

¹H NMR (CDCl₃, 400 MHZ) δ 8.21 (2H, d), 6.44 (2H, dd), 6.26 (2H, d), 4.10 (8H, t), 2.50 (4H, p).

### Synthesis of 1-(6-(azetidin-1-yl)-9-phenyl-3H-xanthen-3-ylidene)azetidin-1-ium chloride

A solution of phenylmagnesium chloride in THF (2M, 0.55 mL) was added to a stirred suspension of 3,6-di(azetidin-1-yl)-9H-xanthen-9-one (0.28 g, 0.91 mmol) in THF (10 mL). After stirring for 1 h, the reaction mixture was diluted with DCM and saturated aqueous NH₄Cl. The layers were separated and the aqueous was extracted three times with DCM. The combined organic layers were dried (MgSO₄), filtered and the solvent was removed in vacuo. The resulting residue was dissolved in DCM and 1 drop of 2 M aqueous HCl was added. The mixture was subsequently concentrated in vacuo and then co-evaporated with firstly MeOH and then with DCM. The crude product was purified by flash chromatography (10 to 20% MeOH in DCM) followed by precipitation of the product from a solution in DCM with EtOAc to give the title compound as a red solid (0.024 g, 7%).

¹H NMR (d6-DMSO, 400 MHZ) δ 7.70-7.66 (3H, m), 7.49-7.47 (2H, m), 7.19 (2H, d), 6.70 (2H, dd), 6.58 (2H, d), 4.28 (8H, t), 2.51 (4H, p).

HRMS (ESI) calcd for C25H23N2O [M]⁺, 367.1810, found 367.1809.

### Example 4-Synthesis of 1-(7-(azetidin-1-yl)-5,5-dimethyl-10-phenyldibenzo[b,e]silin-3 (5H)-ylidene)azetidin-1-ium trifluoroacetate

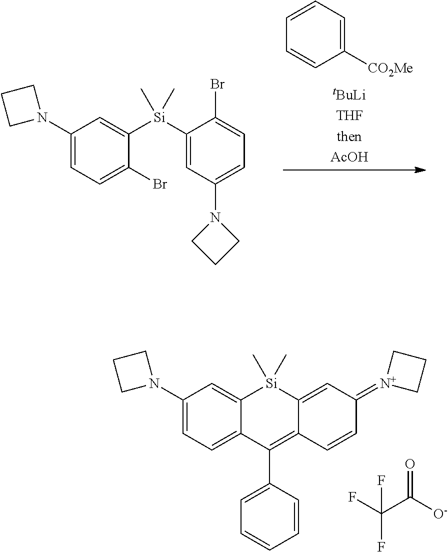 [SMILES: COC(=O)c1ccccc1 ; C[Si](C)(c1cc(N2CCC2)ccc1Br)c1cc(N2CCC2)ccc1Br ; C[Si]1(C)C2=CC(=[N+]3CCC3)C=CC2=C(c2ccccc2)c2ccc(N3CCC3)cc21 ; O=C([O-])C(F)(F)F]

### Synthesis of 1-(7-(azetidin-1-yl)-5,5-dimethyl-10-phenyldibenzo[b,e]silin-3 (5H)-ylidene)azetidin-1-ium trifluoroacetate

A solution of t-BuLi in pentane (1.7 M, 1.96 mL) was added dropwise to a cooled (−78° C.) solution of bis(5-(azetidin-1-yl)-2-bromophenyl)dimethylsilane (0.40 g, 0.83 mmol) in THF (40 mL). After stirring for 20 minutes, the reaction mixture was warmed to −20° C. and a solution of methyl benzoate (0.25 g, 1.83 mmol) in THF (7 mL) was added over 20 minutes, maintaining the internal temperature below −20° C. over the course of the addition. The resulting mixture was allowed to warm to room temperature and was stirred overnight. The following day, saturated aqueous NH₄Cl and water were added and the product was extracted twice with EtOAc. The combined organic layers were washed with brine, then dried (MgSO₄) and filtered and the solvent was removed in vacuo. The resulting residue was dissolved in MeOH (20 mL) and AcOH (0.2 mL) was added. After stirring for 15 minutes, the mixture was concentrated in vacuo. The crude product was initially purified by flash chromatography (1% MeOH in DCM (+1% AcOH) to 15% MeOH in DCM (+1% AcOH). Subsequently, half of the crude product was purified by prep-LC (10-100% ACN/H2O (+0.1% TFA) and concentrated by lyophilisation to furnish the desired product as a blue/red solid (0.011 g)

¹H NMR (d6-DMSO, 400 MHZ) δ 7.61-7.52 (3H, m), 7.30-7.22 (2H, m), 7.05 (2H, d), 6.91 (2H, d), 6.42 (2H, dd), 4.40-4.22 (8H, m), 2.48-2.39 (4H, m), 0.54 (6H, s).

LC/MS (ES+): m/z 409.3 (100%, M⁺).

### Example 5-1-(6-(azetidin-1-yl)-9-(o-tolyl)-3H-xanthen-3-ylidene)azetidin-1-ium

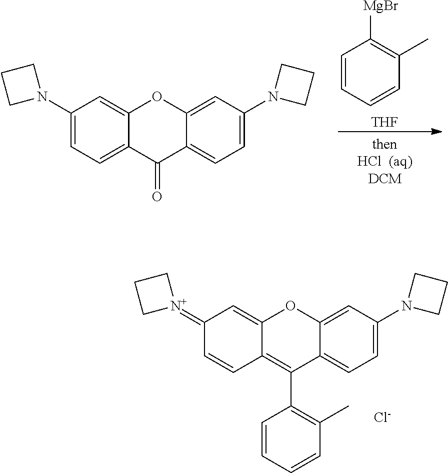

### Synthesis of 1-(6-(azetidin-1-yl)-9-(o-tolyl)-3H-xanthen-3-ylidene) azetidin-1-ium

A solution of o-tolylmagnesium bromide in Et₂O (2M, 1.22 mL) was added to a stirred, pre-heated (50° C.) solution of 3,6-di(azetidin-1-yl)-9H-xanthen-9-one (0.25 g, 0.81 mmol) in THF (12 mL). After stirring at 50° C. for 9 h, the reaction mixture was allowed to cool to room temperature and stirred overnight. The mixture was diluted with DCM and saturated aqueous NH4Cl (10 mL). The layers were separated and the aqueous was extracted three times with DCM. The combined organic layers were dried (MgSO₄), filtered and the solvent was removed in vacuo. The resulting residue was dissolved in DCM and 1 drop of 2 M aqueous HCl was added. The mixture was subsequently concentrated in vacuo and then co-evaporated with firstly MeOH and then with DCM. The crude product was purified by flash chromatography (7 to 20% MeOH in DCM) followed by trituration with EtOAc to give the title compound as a red solid (0.065 g, 19%).

¹H NMR (MeOD, 400 MHZ) δ 8 7.61-7.44 (3H, m), 7.25 (1H, dd), 7.16-7.12 (2H, m), 6.67 (2H, dd), 6.59 (2H, d), 4.36 (8H, t), 2.60 (4H, p), 2.07 (3H, s).

LC/MS (ES+): m/z 381.1 (100%, M⁺).

### REFERENCES

1) Grimm et al., General Synthetic Method for Si-Fluoresceins and Si-Rhodamines. ACS Cent Sci. 2017; 3 (9): 975-985.
2) Macho et al., Chloromethyl-X-rosamine is an aldehyde-fixable potential-sensitive fluorochrome for the detection of early apoptosis. Cytometry. 1996; 25 (4): 333-340.
3) Poot et al.; Analysis of mitochondrial morphology and function with novel fixable fluorescent stains. J Histochem Cytochem. 1996; 44 (12): 1363-72.
4) Grimm et al., A general method to improve fluorophores for live-cell and single-molecule microscopy; Nat Methods. 2015 12 (3): 244-50.
6) EP 3 126 451
7) U.S. Pat. No. 5,686,261

All publications mentioned in the above specification are herein incorporated by reference. Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A method for staining mitochondria, the method comprising:
providing a sample containing mitochondria, and
incubating the sample in a composition comprising a cationic species of formula (I): 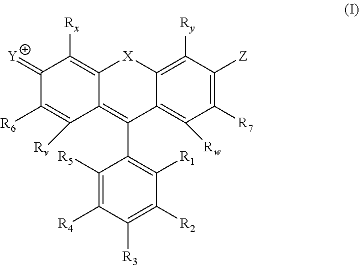
or a solvate, or tautomer thereof; and a counter ion;
wherein:
Y is a substituted or unsubstituted azetidine ring and Z is selected from OR₁₇ or a substituted or unsubstituted azetidine ring;
X is selected from O, S, SO₂, Se, NR₁₂, P(O)R₁₂, CR₁₃R₁₄, SiR₁₃R₁₄, Te, and GeR₁₃R₁₄;
R₁, R₂, R₃, R₄, and R₅ are each independently selected from H, C₁ to C₈ alkyl, OR₁₅, C(O)OR₁₆, NHCOR₁₅, CONHR₁₅ and halo;
Rᵥ, R_{w}, Rₓ, R_{y}, R₆, R₇ are each independently selected from H, C₁ to C₈ alkyl and halo;
R₁₂, R₁₃, R₁₄, and R₁₅ are each independently selected from H, C₁ to C₈ alkyl, optionally substituted aryl or optionally substituted heteroaryl;
R₁₆ is selected from C₁ to C₈ alkyl, optionally substituted aryl or optionally substituted heteroaryl, and
R₁₇ is selected from H, C₁ to C₈ alkyl, optionally substituted aryl or optionally substituted heteroaryl.

2. A method as claimed in claim 1, wherein at least one of Y and Z is a substituted or unsubstituted azetidine group of formula: 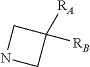
wherein R_{A} and R_{B} are independently selected from H, halo, C₁ to C₈ alkyl, optionally substituted aryl or optionally substituted heteroaryl.

3. A method as claimed in claim 1, wherein the cationic species is of formula (II): 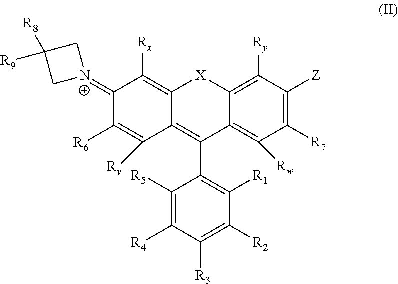
wherein R₈ and R₉ are independently selected from H, halo, C₁ to C₈ alkyl, optionally substituted aryl or optionally substituted heteroaryl.

4. A method as claimed in claim 1, wherein the counter ion is a biologically compatible counterion.

5. A method as claimed in claim 1, wherein the counter ion is selected from halide, carboxylate, oxalate, sulfate, alkanesulfonate, arylsulfonate, phosphate, perchlorate, trifluoroacetate, tetrafluoroborate, tetraphenylboride, hexafluorophosphate, nitrate and anions of aromatic or aliphatic carboxylic acids.

6. A method as claimed in claim 1, wherein incubating the sample is for a predetermined time, in the range 10 mins to 2hours and at a predetermined temperature, in the range 20° C. to 39° C.

7. A method as claimed in claim 1, wherein the cationic species is of formula (III): 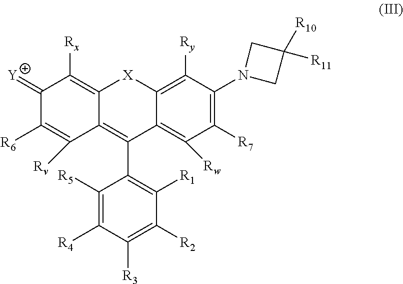
wherein R₁₀ and R₁₁ are independently selected from H, halo, C₁ to C₈ alkyl, optionally substituted aryl or optionally substituted heteroaryl.

8. A method as claimed in claim 1, wherein the cationic species is of formula (IV):

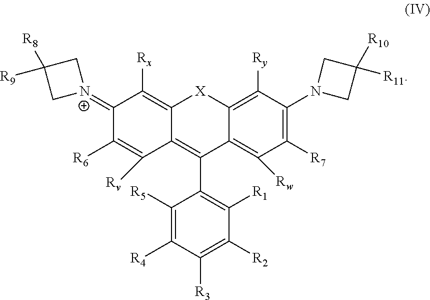

9. A method as claimed in claim 1, wherein R₁, R₂, R₃, R₄, R₅, R₆, R₇, R₈, R₉, R₁₀, and R₁₁ are independently H, fluoro or chloro.

10. A method as claimed in claim 1, wherein R₁ and/or R₅ are C₁ to C₈ alkyl.

11. A method as claimed in claim 10, wherein R₁ and/or R₅ are methyl.

12. A method as claimed in claim 1, wherein the composition further comprises at least one organic solvent.

13. A method as claimed in claim 12, wherein the at least one organic solvent is selected from DMSO, acetone, dimethylformamide, acetonitrile, dioxane, and THF.

14. A method as claimed in claim 1, wherein the sample containing mitochondria comprises a tissue sample.

15. A method as claimed in claim 1, wherein the sample containing mitochondria is a plant, animal or fungal tissue sample, a sample of plant, animal or fungal cells or isolated plant, animal or fungal mitochondria.

16. A method as claimed in claim 1, wherein the sample containing mitochondria comprises a sample containing live mitochondria and/or a sample containing mitochondria in live cells.

17. A method as claimed in claim 1, wherein the sample containing mitochondria does not contain fixed cells.

18. A method as claimed in claim 1, wherein the concentration of the cationic species in the composition is in the range 10 nM to 1 μM.

19. A method as claimed in claim 1, wherein the cationic species of formula (I) is selected from species of formulae: 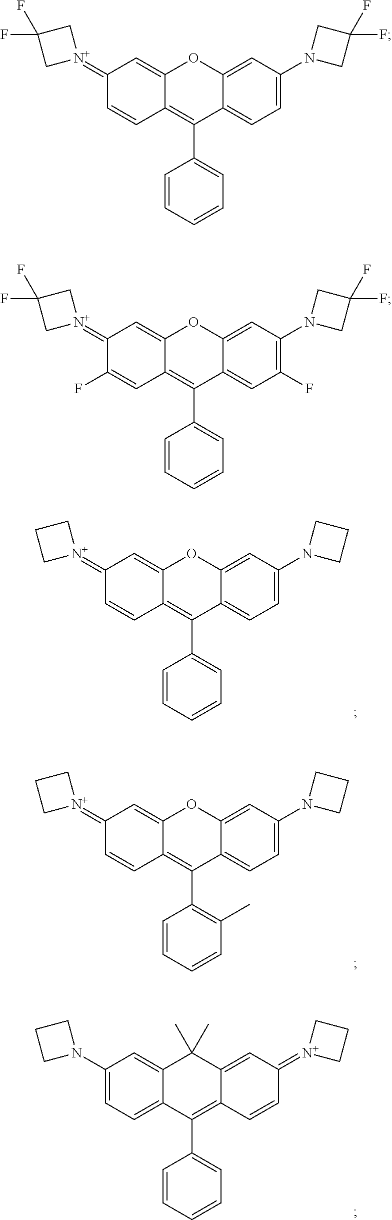 [SMILES: FC1(F)CN(c2ccc3c(-c4ccccc4)c4ccc(=[N+]5CC(F)(F)C5)cc-4oc3c2)C1 ; Fc1cc2c(-c3ccccc3)c3cc(F)c(=[N+]4CC(F)(F)C4)cc-3oc2cc1N1CC(F)(F)C1 ; c1ccc(-c2c3ccc(=[N+]4CCC4)cc-3oc3cc(N4CCC4)ccc23)cc1 ; Cc1ccccc1-c1c2ccc(=[N+]3CCC3)cc-2oc2cc(N3CCC3)ccc12 ; CC1(C)C2=CC(=[N+]3CCC3)C=CC2=C(c2ccccc2)c2ccc(N3CCC3)cc21] 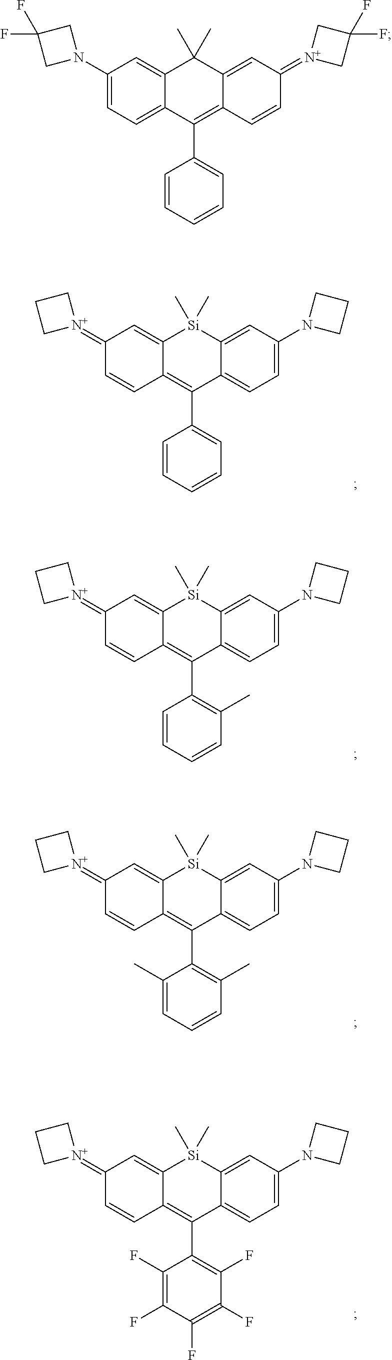 [SMILES: CC1(C)C2=CC(=[N+]3CC(F)(F)C3)C=CC2=C(c2ccccc2)c2ccc(N3CC(F)(F)C3)cc21 ; C[Si]1(C)C2=CC(=[N+]3CCC3)C=CC2=C(c2ccccc2)c2ccc(N3CCC3)cc21 ; Cc1ccccc1C1=C2C=CC(=[N+]3CCC3)C=C2[Si](C)(C)c2cc(N3CCC3)ccc21 ; Cc1cccc(C)c1C1=C2C=CC(=[N+]3CCC3)C=C2[Si](C)(C)c2cc(N3CCC3)ccc21 ; C[Si]1(C)C2=CC(=[N+]3CCC3)C=CC2=C(c2c(F)c(F)c(F)c(F)c2F)c2ccc(N3CCC3)cc21] 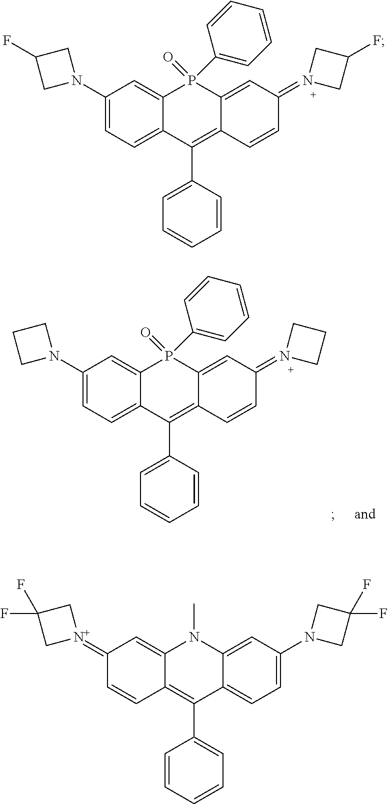
or solvates, or tautomers thereof.

20. A method of analysing mitochondria, the method comprising:
staining a sample of mitochondria using a method as claimed in claim 1,
illuminating the stained sample using light of an appropriate wavelength to fluoresce the compound, and
observing or imaging a magnified image of the sample.

21. A method as claimed in claim 20, wherein the appropriate wavelength is in the range 400 nm to 800 nm.

22. A method of detecting a mitochondrial condition, the method comprising staining a sample of mitochondria as claimed in claim 1.

23. A method as claimed in claim 22, wherein the sample of mitochondria is a plant, animal or fungal tissue sample, a sample of plant, animal or fungal cells or isolated plant, animal or fungal mitochondria.

24.-25. (canceled)
